(19) United States
(12) Patent Application Publication
(10) Pub. No.: US 2012/0148650 A1
(43) Pub. Date: Jun. 14, 2012

(54) MULCH-MODIFYING SPRAYABLE LATEX

(75) Inventors: Michael R. Hogatt, Naples, FL (US); Roger W. Faulkner, Cambridge, NY (US); James L. Zwynenberg, Queensbury, NY (US)

(73) Assignee: EnviroHold Inc.

(21) Appl. No.: 13/213,964

(22) Filed: Aug. 19, 2011

Related U.S. Application Data
(60) Provisional application No. 61/421,775, filed on Dec. 10, 2010.

(51) Int. Cl. A01N 25/26; A61L 9/01; A01N 59/16; C09D 131/04; C08K 5/17; C08K 3/22; C09D 129/04; A01N 61/00; A01P 17/00; B82Y 30/00

(52) U.S. Cl. 424/405; 514/772.3; 424/76.1; 424/641; 524/524; 524/186; 524/431; 524/432; 524/503; 977/773

(57) ABSTRACT

This disclosure concerns improved mulch holding, mulch preserving, and/or mulch pigmenting sprayable latex, and its application to preserving the appearance of decorative mulch, either by fixing decorative mulch in place more effectively than prior mulch-holding latex, by protecting the mulch from biological or UV degradation, by pigmenting or staining the mulch, or some combination of two or more of these characteristics.

### CROSS-REFERENCE TO RELATED APPLICATION

This application claims the benefit of the filing date of U.S. Provisional Application Ser. No. 61/421,775 filed Dec. 10, 2010 and entitled “Mulch-Modifying Sprayable Latex”, incorporated by reference herein.

### FIELD

The invention pertains to spraying of diluted latex to stabilize mulch or freshly tilled soil against washing away in a rainstorm or due to high wind. More particularly, the invention pertains to spraying practiced in several different ways.

### BACKGROUND

Spraying of diluted latex to stabilize mulch or freshly tilled soil against washing away in a rainstorm or due to high wind is practiced in several different ways. Prior art patents specifically mention PVAc (polyvinyl acetate), EVA (ethylene-vinyl acetate copolymer), SBR (styrene-butadiene rubber), and acrylic latex (copolymers of ethyl and/or butyl acrylate), all of which form water-resistant polymer films. The prior art also mentions several water-soluble or highly water-swellable polymers such as polyacrylamide and several natural gum polymers.

A commercial example of a company practicing the application of latex to stabilize decorative mulch is RS Industrial, Inc. which currently sells a dilutable PVAc latex, brand name MulchHold, see website “mulchhold.com.” The instructions for MulchHold (from their website) suggests a dilution between 2:1 to 4:1 with water. Testing of MulchHold as supplied showed a 55-58% solids PVAc latex, essentially the same as Rovace 117 from Rohm & Haas. The solids content after 4:1 dilution is approximately 11.5% (this is the standard polymer % content adopted for many, but not all of the experiments herein), the same as is recommended by U.S. Pat. No. 3,475,435. U.S. Pat. No. 3,475,435 describes binding vermiculite or a similar planting medium together so as to be resistant to being displaced by wind or rain. The commercial marketing and sales of MulchHold shows use of PVAc latex to stabilization of decorative mulch.

Three major synthetic polymers that are mentioned in the context of film-forming sprayable soil amendments are polyvinyl acetate (PVAc), mentioned in U.S. Pat. Nos. 3,475,435; 4,283,445; 6,622,428, for example; polyacrylates, used for example in U.S. Pat. No. 7,289,430, and SBR elastomer latex (U.S. Pat. No. 4,071,400). Of these three, PVAc is the most readily biodegradable. Polyacrylates have the highest intrinsic UV resistance among these polymers.

U.S. Pat. No. 4,283,445 describes forming a mulch blanket by spraying a polyvinyl acetate (PVAc) latex (Curasol™ from Hoechst is specifically mentioned) onto a thick bed of mulch and after drying, peeling the resultant bonded mulch blanket off the surface of the pile. No dilution, viscosity, nor solids content is specified.

A 1996 report of the UN Food and Agriculture Organization “Land husbandry—Components and strategy” (ISBN 92-5-103451-6) also mentions Curasol™ from Hoechst for soil stabilization (against erosion by heavy rain) by spraying, but in this case it was sprayed directly on tilled soil, rather than onto mulch. In this instance, Curasol was diluted: 60 grams of Curasol diluted in 1 liter of water per square meter of soil. Curasol PVAc latex does not appear to be manufactured by Hoechst if at all.

U.S. Pat. No. 3,475,435 claims a preferred solids content of 10-15%, or more preferably 11-13% for the diluted PVAc latex to be sprayed onto the vermiculite. This patent also mentions other “granular materials”, such as sawdust, perlite, rice hulls, or peanut shells; in all cases, the purpose of the granular material is to provide a desirable medium for seed germination and the first stage of growth. No mention is made of use in landscaping. This patent is cited in the recent U.S. Pat. No. 7,765,735 by Samuel P. Carelli (Aug. 3, 2010), which does disclose the application of PVAc latex to stabilizing decorative mulch. This patent also refers to an earlier provisional patent, 61/190,644, “Mulch tech: a system of application and adhesion for the semi-permanent anchoring of mulch with additives to prevent smoldering and weed control;” this published provisional application mentions the concept of dying mulch in place either to return it to its initial color, or to enhance its color.

It is also known to use other forms of polymers, such as water-soluble or water-swellable polymers in the art; for examples, U.S. Pat. Nos. 6,835,781; 7,407,993; 7,666,923; 7,73,0632, describe use of copolymers of acrylamide (PAM) as both bonding agents and to absorb and hold water, both to water the plants and for fire resistance; the resultant swelling and deswelling in response to rain and humidity changes make PAM a poor adhesive for holding mulch together. Pure polyacrylamide is truly water soluble, and the copolymers of PAM are either water soluble or highly water swellable, which makes PAM difficult to spray (because it thickens the water). By contrast film-forming latexes such as PVAc, SBR and polyacrylates are easily diluted in water.

Several prior patents on soil or mulch binders mention multiple different binders, but also bring in additional features, such as clay added to the latex (U.S. patent application 2007/0000167), or fibers (U.S. Pat. No. 7,681,353). There are also numerous naturally occurring binders mentioned in patents, such as psillium seed husks, agar-agar, starches, shellac, terpene polymers, and plant-derived proteins.

The art includes the general concept of adding major plant nutrients (nitrogen, phosphorous, and potassium), insecticides, fungicides, and herbicides to a mulch-binding latex. Addition of water soluble modified cellulose ethers, such as hydroxypropyl methyl cellulose, to provide thixotropy and anti-settling activity is mentioned in prior art patents (for example U.S. Pat. No. 4,071,400), but the inventors are not aware of these additives being claimed to improve either shear stability or freeze-thaw stability.

U.S. Pat. No. 7,754,801 on UV stabilizers for wood describes latex systems that contain multiple and synergistic UV-blocking and/or UV-protective components, including various organic materials such as hindered amine light stabilizers (HALS) and UV absorbers, combined with various fine particle size metal oxides that strongly absorb UV, such as zinc oxide, iron oxide, cerium oxide, and titanium oxide. In the case of transparent iron oxides, a staining effect on the wood substrate is also noted. This patent does not contemplate nor describe use of the protective spray to bind mulch particles in place, nor to slow biodegradation of the mulch, nor to supply micronutrients, for example.

### BRIEF DESCRIPTION OF THE DRAWINGS

FIG. 1 shows a graph of the initial viscosity of latex blends;

FIG. 2 shows a graph of polymer content of diluted latex;

FIG. 3 shows thermogravimetric analysis of PR-275 Latex;

FIG. 4 shows thermogravimetric analysis of MulchHold concentrate;

FIG. 5 show thermogravimetric analysis of Rvace 117 PVAc;

FIG. 6 shows the UV absorbance of various emulsion C systems;

FIG. 7 shows a graph of viscosity vs shear rate for various mixtures; and

FIG. 8 shows DSC Curve for emulsions.

### SUMMARY OF THE INVENTION

The present invention comprises improved mulch holding, mulch preserving, and/or mulch pigmenting sprayable latex, and its application to preserving the appearance of decorative mulch, either by fixing decorative mulch in place more effectively than prior mulch-holding latex, by protecting the mulch from biological or UV degradation, by pigmenting or staining the mulch, or some combination of two or more of these characteristics. The improvements over prior art mulch holding sprayable latex products include one or more of these novel features:
improved UV resistance of the polymer film itself;
sunscreen effect: protecting the color of expensive mulch such as cedar chips which are subject to fading due to UV exposure;
improved latex stability against freeze-thaw;
improved resistance to creaming or separation in storage;
improved stability during dilution;
controlled release of micronutrients;
slowed biodegradation;
controlled release of natural pest controls, herbicides, cat repellants, mosquito repellants, odors, etc.;
controlled penetration into porous mulch;
re-pigmentation of mulch: returning sun-faded mulch to a simulated original color;
pigmentation of the mulch to a selected aesthetically pleasing shade;
use of high strength polyurethane latex to create strong bonding between next neighbor mulch particles, so that less latex is required to bind the mulch together;
use of micro-emulsion based primer coats to optimize the overall properties of the latex-modified mulch; said microemulsions are useful for staining and preserving mulch, and can be applied either before or after application of the mulch to the ground; the thus treated mulch particles may or may not be subsequently bonded together by a second adhesive latex.

Improving the UV resistance of the film-forming polymer used as the basis for the novel sprayable latex can most conveniently be accomplished by adding a hindered amine light stabilizer (HALS). Addition of HALS to a PVAc or EVA latex can increase the UV resistance of PVAc or EVA substantially, so that the PVAc or EVA films can achieve nearly equivalent life in sunlight to polyacrylates. Alternatively, HALS can be added to acrylic latex to achieve an even longer life in direct sunlight. The addition level of HALS to a latex can also be used to target a specific survival time of the mulch coating in direct sunlight.

Adding HALS to the film-forming synthetic latex polymer protects the synthetic polymer per se, but does little to protect the substrate mulch particle from bleaching under UV exposure due to sunlight. Some expensive mulches, like cedar chips or redwood bark for example, are highly valued for their color, which fades under direct sunlight. The rate of this UV-induced fading can be reduced imparting to the latex a sunblock functionality. This can be done for example by adding UV absorbers to the latex, such as zinc oxide, iron oxide, titanium dioxide, cerium oxide, various cinnamate esters, para-aminobenzoic acid (PABA), and/or benzophenone and benzotriazole derivatives. Where metal oxide particles are used to block UV, it is highly desirable that they be small enough, and well dispersed enough, that the resulting film is transparent or translucent. Transparent iron oxides also have a dying effect, and can be used to create a range of shades from yellow to red, and are far more UV stable than the naturally occurring colors in plant-derived mulch (such as cedar chips or redwood bark).

Another desirable feature of the present invention is stability against freeze-thaw. This is significant in locations where the product may freeze during storage in an unheated garage or shed over the winter. The subject invention includes several different methods that can be used to improve freeze-thaw stability of a latex, including formulation of the latex with known water soluble or water dispersible polymers that can improve freeze-thaw stability, certain colloidal dispersions, including various microemulsions, and various non-ionic surfactants containing polymeric hydrophiles with molecular weight >1000 daltons.

Another desirable feature of the present invention is stability against creaming or separation in storage. This is significant in determining the shelf life of a latex, because creaming or separation often precedes coagulation. The subject invention includes several different methods that can be used to improve resistance to creaming or separation in storage of an improved mulch modifying sprayable latex, including a wide variety of additives that impart a gel strength to the water-rich matrix phase of the latex which is high enough to prevent sedimentation of the polymer latex particles in the improved mulch modifying sprayable latex.

Another desirable feature of the present invention is improved stability during dilution. Most latexes can be diluted, but in practice some latexes must be carefully diluted to avoid partial coagulation, while others can be “splash blended” with added water with little or no coagulation, regardless of dilution procedures. Since it is difficult to get consumers of a product to follow directions, it is highly desirable to impart to the improved mulch modifying sprayable latexes of this invention the property of being easily dilutable, whether the latex is poured into water or the converse. The subject invention includes several different methods that can be used to improve dilutability of an improved mulch modifying sprayable latex, including selecting non-ionically stabilized latex, adding to the selected latex non-ionic emulsifiers and/or stabilizers, adding to the selected latex known water soluble or water dispersible polymers that improve latex stability, including especially certain polymers that form protective layers on latex polymer droplets, such as kelzan gum, particular grades of Cabopol™ polymers from Lubrizol that contain grafted hydrophobic groups (such as Carbopol® ETD 2623), or various Pluronic™ surfactants from BASF (block copolymers of poly[ethylene oxide] with poly[propylene oxide]) in particular. Certain colloidal dispersions, including various microemulsions, and various non-ionic surfactants containing polymeric hydrophiles with molecular weight >1000 daltons are also helpful in preventing partial coagulation during dilution, such as castor oil ethoxylates with polyethylene oxide (PEO) hydrophiles containing more than 33 polymerized ethylene oxide units.

Another desirable feature of the present invention is improved controlled release of secondary nutrients and micronutrients. Prior art patents have disclosed the concept of primary nutrient incorporation into sprayable latex. Primary nutrients include nitrogen, phosphorous, and potassium. Those nutrients are the major components of most fertilizers and while important they do not make much sense to incorporate into a mulch modifying sprayable latex, for two reasons. First, these fertilizer components are relatively soluble and so will generally leach out of the sprayed mulch before the end of the useful life of the mulch. Secondly, these primary fertilizers, especially nitrogen, accelerates biodecay of lignocellulosic mulch, which is not desirable for a decorative mulch. There are other important plant nutrients that are vital yet secondary in the sense that less of these are needed than the primary nutrients, such as magnesium (required for chlorophyll, the protein that captures sunlight), iron, calcium, and zinc (used by all cells during replication). A third class of nutrients are the micronutrients, of which there are many, including selenium, copper, manganese, and chromium for example; these are also required for healthy plants. Because the concentrations of secondary and micro nutrients required for healthy plants is much less than the amount of primary nutrients required by healthy plants, it is relatively more practical to incorporate secondary and micronutrients into mulch modifying sprayable latex, and one aspect of the present invention is to do so.

Another desirable feature of the present invention is to slow down the biodegradation of the mulch. One way to do so in principle is to treat the mulch with poisonous substances, such as creosote, pentachlorophenol, or copper-chrome-arsenic for example, but such an approach is not environmentally or aesthetically acceptable. Certain secondary or micronutrients, such as zinc in particular, are known to be “biostatic,” inhibiting biodecay without environmental toxic effects. Thus, incorporation of zinc oxide nanoparticles into an improved mulch modifying sprayable latex has multiple desirable effects, including protection of decorative mulch particles from UV bleaching, slowing down biodecay of the mulch particles, and leaching a vital secondary nutrient into the soil beneath the mulch.

Another desirable feature of the present invention is to provide for controlled release of pesticides, fungicides, herbicides, cat repellants, mosquito repellants, desirable odors, etc. Controlled release of useful pest control chemicals have been mentioned in some prior art patents, but specifically chemicals that are useful as cat repellants, mosquito repellants, and desirable odors have not been mentioned in prior art patents to our knowledge, and so are claimed as novel for purpose of this invention. Specific natural pest control materials, such as capsaicin, tea tree oil, eucalyptus oil, citronella, and neem oil have also not been mentioned in prior art patents, and are claimed as part of this invention.

Another desirable feature of the present invention is to provide controlled penetration into porous mulch. For one thing, the degree to which a latex penetrates into porous mulch determines the bond strength of the polymer film to the mulch and the bond strength between mulch particles. Efficient penetration of latex into the mulch particles would be favored for optimum bonding between the polymer film and the mulch particle, and also where inhibition of biodecay of the mulch particle is one goal of the application of the latex spray. However, efficient penetration of the mulch particles tends to leave less latex on the surface of the mulch particles, which leads to less bonding between the mulch particles. It has been discovered that the degree to which the latex spray penetrates into the mulch particles can be controlled via the addition of wetting agents, dispersants, and/or microemulsion latex. For optimum penetration into latex particles, a diluted latex formulation comprising a non-ionically stabilized microemulsion is especially desirable.

Another desirable feature of the present invention is to provide a method to stain a mulch a desired color, or to re-pigment mulch so as to return sun-faded mulch to a simulated original color. High surface area iron oxide pigments known in the paint industry as “transparent iron oxides” (BET surface area >80 m²/gram) are especially suitable, because they also serve as micronutrients an UV absorbers. Colors ranging from yellow to red are available for high surface area iron oxide pigments.

The improved mulch modifying sprayable latex of this invention may also comprise high strength polyurethane (PU) latex. As used here, “PU latex” includes any urethane polymer dispersion in water regardless of how it is formed. Since one particular application of the improved mulch modifying sprayable latex of this invention is to bond mulch particles together into a mat, the high strength of PU latex after drying is highly desirable, because a thinner coating on the mulch particles is needed to achieve the desired bond strength between the mulch particles.

The improved mulch modifying sprayable latex of this invention may also comprise micro-emulsion based primer coats to optimize the overall properties of the latex-modified mulch; said microemulsions are useful for staining mulch as mentioned previously, but such compositions are also useful for preserving mulch. Such microemulsion-based primer coats tend to penetrate more deeply into mulch particles than ordinary latex polymers, and can be applied either before or after application of the mulch to the ground; the thus treated mulch particles may or may not be subsequently bonded together by a second adhesive latex.

### EMBODIMENTS OF THE INVENTION

Improving and/or controlling the UV resistance of the film-forming polymer is a desirable feature for the novel sprayable latex of the present invention. UV degradation of the polymer causes yellowing, and sometimes flaking of the degraded polymer. Yellowing is more pronounced in PVAc and styrene-containing latex than in EVA or polyacrylates. UV degradation resistance can be improved by adding either a UV-blocking additive (a sunscreen) or a hindered amine light stabilizer (HALS). HALS do not absorb UV radiation, but act to inhibit degradation of the polymer. One set of examples of HALS are derivatives of 2,2,6,6-tetramethyl piperidine, which are extremely efficient stabilizers against light-induced degradation of most polymers. They slow down the photochemically initiated degradation reactions, in a similar way to antioxidants. Addition of HALS to a PVAc or EVA latex can increase the UV resistance substantially, so that the PVAc or EVA latex can achieve equivalent life in sunlight to polyacrylates. Alternatively, HALS can be added to acrylic latex to achieve an even longer life in direct sunlight. Alternatively, HALS can be used determine a preferred survival time of the polymer film, followed by rapid UV degradation.

Adding HALS to the film-forming synthetic latex polymer protects the synthetic polymer per se from UV degradation, but does little to protect the substrate mulch particle from bleaching under UV exposure due to sunlight. Some expensive mulches, like cedar chips or redwood bark for example, are highly valued for their color, which fades under direct sunlight. The rate of this UV-induced fading can be reduced by imparting to the latex a sunblock functionality. This can be done for example by adding UV absorbers to the latex, such as benzophenone and benzotriazole derivatives, various cinnamate esters, para-aminobenzoic acid (PABA), zinc oxide, iron oxide, cerium oxide, and/or titanium dioxide. In the case of zinc oxide, iron oxide, cerium oxide, and/or titanium dioxide particles, it is highly desirable that the particles be small enough so as not to scatter light. U.S. Pat. No. 7,754,801 describes such a latex-based sunscreen for wood, but does not mention application to a binder latex for decorative mulch. It is also critically important that a stable mixture with the latex can be formed. Latex stability is hard to achieve for systems containing zinc oxide and cerium oxide particles in particular. Because of that, several types of coated zinc oxide particle are available, including zinc stearate or zinc propionate coatings (used mainly in rubber), and silicone coatings (example: Z-Cote from BASF, used in sunscreen lotions and transparent coatings). In the case of cerium oxide particles, particle coatings based on boron nitride have been found to be effective to improve latex stability in blends containing the coated particles compared to pure cerium oxide particles. A synergistic blending of multiple different types of sunscreens is anticipated in U.S. Pat. No. 7,754,801. Combining the function of mechanically bonding the mulch particles together while simultaneously protecting them from UV bleaching was not anticipated in U.S. Pat. No. 7,754,801. Zinc oxide, iron oxide, and titanium dioxide are preferred over cerium oxide from a pollution point of view for improving the UV resistance of the film-forming polymer within the novel sprayable latex. Iron oxide can be in the form of transparent iron oxides that vary in color between yellow to red.

Another inventive way to add sunblock functionality to a mulch-treating dilutable latex is to use naturally occurring nanoparticles found in the sap of some climbing vines, such as ivy. Ivy nanoparticles may protect skin from UV radiation at least four times better than the metal-based sunblocks found on store shelves today. The present invention illustrates the formulation of these or similar plant-derived UV-protective nanoparticles with latex polymers for protection of mulch against UV bleaching.

Another desirable feature of the present invention of the present invention is stability against freeze-thaw. This is significant in locations where the product may freeze during storage in an unheated garage or shed over the winter. The subject invention includes several different methods that can be used to improve freeze-thaw stability of a latex, including formulation of the latex with known water soluble or water dispersible polymers that can improve freeze-thaw stability by imparting to the water a sufficient gel strength to slow or prevent Brownian motion of the polymer droplets in the latex. Particular examples of water-soluble polymers that can improve freeze-thaw stability include various natural gums such as guar or kelzan gum, partially synthetic polymers such as hydroxyethyl cellulose, and synthetic hydrophilic polymers such as polyvinyl alcohol (PVOH). Water dispersible polymers include lightly crosslinked poly(acrylic acid) based polymers such as Cabopol™, Ultez™, or Pemulan™ copolymers (all from Lubrizol, Inc.). Certain colloidal dispersions, including various nanosized clays and microemulsions (latex in which the maximum size of latex particles is <200 nm, with most particles smaller than 120 nm) also improve freeze-thaw stability. Various non-ionic surfactants containing polymeric hydrophiles with molecular weight >1000 daltons improve freeze thaw stability by surrounding each latex particle with a hydrophilic polymer shell (“steric stabilization”). A particularly useful microemulsion both for improving freeze thaw stability and shear stability is Joncryl™ 624, a styrene-butyl acrylate copolymer, which is anionically stabilized by a special surfactant that leads to latex particles between 80-120 nm. (This is around one tenth the diameter of typical latex polymers such as the PVAc latexes that have previously been used for bonding mulch particles together.) Joncryl™ 624 is also believed to include non-ionic surfactants as well. A particularly useful group of non-ionic surfactants containing polymeric hydrophiles are the castor oil ethoxylates and/or fatty alcohol ethoxylates containing hydrophilic groups comprised of 20-50 polymerized ethylene oxide units.

Another desirable feature of the present invention of this invention is stability against creaming or settling of the latex particles in storage. This is significant in determining the shelf life of a latex, because creaming or separation often precedes coagulation. The subject invention includes adding viscosity-building water soluble polymers to the latex compounds of this invention to improve resistance to creaming or separation in storage of an improved mulch modifying sprayable latex, including a wide variety of additives that impart a gel strength to the water-rich matrix phase of the latex which is high enough to prevent sedimentation or creaming of the polymer latex particles in the improved mulch modifying sprayable latex. Some of the same water soluble polymers which improve freeze-thaw stability also work to prevent creaming (for polymers that are less dense than water), or settling (for polymers that are more dense than water). Effective polymers to prevent creaming or settling of the latex include lightly crosslinked poly(acrylic acid) polymers such as Cabopol™ 941 or Cabopol™ 981, or Ultez™ 20, or Pemulan copolymers (all from Lubrizol, Inc.), certain colloidal dispersions, including various nanosized clays and polymer microemulsions (latex in which the maximum size of latex particles is <200 nm, with most particles smaller than 120 nm), various modified cellulose ethers such as hydroxyethyl cellulose or carboxymethyl cellulose, and various natural gums such as kelzan gum.

Another desirable feature to impart to improved mulch modifying sprayable latex of this invention is improved stability during dilution. Most latexes can be diluted, but in practice some latexes must be carefully diluted to avoid partial coagulation, while others can be “splash blended” with added water with little or no coagulation, regardless of dilution procedures. Since it is difficult to get consumers of a product to follow directions, it is highly desirable to impart to the improved mulch modifying sprayable latexes of this invention the property of being easily dilutable, whether the latex is poured into water or the converse. The subject invention includes several different methods that can be used to improve dilutability of an improved mulch modifying sprayable latex, including selecting non-ionically stabilized latex, or adding non-ionic emulsifiers to the latex formulation prior to dilution, and appropriate selection of viscosity-modifying polymers. Splash blendability is inhibited by crosslinked polymer hydrogels like Carbopol or Pemulan than it is by completely water soluble polymers like hydroxyethyl cellulose or polyvinyl alcohol.

Water dispersible polymers that improve latex stability include certain polymers that form protective layers on latex polymer droplets, such as kelzan gum, particular grades of Cabopol™ or Pemulan™ polymers from Lubrizol that contain grafted hydrophobic groups (such as Carbopol® ETD 2623 or Pemulan TR-2), or various Pluronic™ surfactants from BASF (block copolymers of poly[ethylene oxide] with poly[propylene oxide]) in particular. Certain colloidal dispersions, including various microemulsions and various non-ionic surfactants containing polymeric hydrophiles with molecular weight >1000 daltons are also helpful in preventing partial coagulation during dilution, high shear mixing, or freeze-thaw cycling.

Another desirable feature of the present invention is improved controlled release of secondary nutrients and micronutrients. The prior art has disclosed the concept of primary nutrient incorporation into sprayable latex. Primary nutrients include nitrogen, phosphorous, and potassium. Those nutrients are the major components of most fertilizers and while important they do not make much sense to incorporate into a mulch modifying sprayable latex, for two reasons. First, these fertilizer components are relatively soluble and so will generally leach out of the sprayed mulch before the end of the useful life of the mulch. Secondly, these primary fertilizers, especially nitrogen, accelerates biodecay of lignocellulosic mulch, which is not desirable for a decorative mulch. There are other important plant nutrients that are vital yet secondary in the sense that less of these are needed than the primary nutrients, such as magnesium (required for chlorophyll, the protein that captures sunlight), iron, calcium, and zinc (used by all cells during replication). A third class of nutrients are the micronutrients, of which there are many, including selenium, copper, manganese, molybdenum, and chromium for example; these are also required for healthy plants. Because the concentrations of secondary and micro nutrients required for healthy plants is much less than the amount of primary nutrients required by healthy plants, it is relatively more practical to incorporate secondary and micronutrients into mulch modifying sprayable latex, and one aspect of the present invention is to do so.

Another desirable feature of the present invention is to slow down the biodegradation of the mulch. One way to do so in principle is to treat the mulch with poisonous substances, such as creosote, pentachlorophenol, or copper-chrome-arsenic for example, but such an approach is not environmentally or aesthetically acceptable. Certain secondary or micronutrients, such as zinc in particular, are known to be “biostatic,” inhibiting biodecay without environmental toxic effects. Thus, incorporation of zinc oxide nanoparticles into an improved mulch modifying sprayable latex has multiple desirable effects, including protection of decorative mulch particles from UV beaching, slowing down biodecay of the mulch particles, and leaching a vital secondary nutrient into the soil beneath the mulch.

Another desirable feature of the present invention is to provide for controlled release of pesticides, fungicides, herbicides, etc. Controlled release of pesticides, fungicides, and herbicides have been mentioned in the prior art, but specific chemicals that are useful as cat repellants, mosquito repellants, and desirable odors have not been mentioned in prior art patents to our knowledge, and so are claimed as novel for purpose of this invention. Specific natural pest control materials, such as capsaicin, tea tree oil, citronella oil, eucalyptus oil, and neem oil are not believed to have been mentioned in prior art patents, and are claimed as part of this invention. By incorporating these chemicals into a polymer film, a controlled release rate occurs which can maintain an effective repellency or toxicity to pests for much longer than if the chemicals were sprayed directly onto the mulch.

Another desirable feature of the present invention is to provide controlled penetration into porous mulch. For one thing, the degree to which a latex penetrates into porous mulch affects the bond strength of the polymer film to the mulch and the bond strength between mulch particles, because latex that penetrates deeply into the mulch particle is not available at the surface of the mulch particle to form the bond between neighboring mulch particles. Some penetration of latex into the mulch particles is favored for optimum bonding between the polymer film and the mulch particle, however. If a goal of application of the latex is the inhibition of biodecay of the mulch particle, then deep penetration of the latex into the mulch particle is desirable, as can best be obtained with microemulsions (this is mainly relevant for primer coats). Efficient penetration of the mulch particles tends to leave less latex on the surface of the mulch particles, which leads to less bonding between the mulch particles. It has been discovered that the degree to which the latex spray penetrates into the mulch particles can be controlled via the addition of viscosity-building water soluble polymers as previously recited, wetting agents such as sodium lauryl sulfate or various other ionic or non-ionic surfactants, dispersants, and/or microemulsion latex. Less penetration into mulch particles occurs with relatively high levels of water-soluble polymer such as PVOH in a PVAc latex compared to that obtained using relatively low levels of stabilization, as would be obtained by simply diluting a commercial 57% solids PVAc latex such as Rovace 117 from Rohm & Haas to the desired nominal 12% solids use level. Enough PVOH or other water-soluble or water-dispersible polymer should be present in the sprayable 11.5% polymer dispersion so that the viscosity is >70 centipoise (as measured by Brookfield viscometer at 30 RPM) at the dilution used for spraying. This appears to control penetration of the latex into cedar chips to an optimum extent for bonding.

It is also possible and desirable in some cases to first spray the mulch with a primer/sealant formulation comprising a highly penetrating latex formulation based on microemulsion latex and optimized for sunscreen and/or coloring properties and slowing biodecay. By blocking the pores of the mulch, the primer/sealant formulation prevents the second mulch binding latex from penetrating into the mulch to a great extent, and therefore allows for less of the second latex to be applied with lower penetration while still creating good bonding between neighboring mulch particles to stabilize the much bed.

Another desirable feature of the present invention is to provide a method to stain a mulch a desired color, or to re-pigment mulch so as to return sun-faded mulch to a simulated original color. For staining, it is optimal that the colorant particles be stable to UV light and small enough to not scatter light in the visible range to a significant extent, and to penetrate into small pores in the mulch to carry the pigment particles into the porous mulch particles, but not too deeply where the pigmenting effect will be wasted. Transparent iron oxide pigments with nitrogen BET surface area >90 m2/gram are highly desirable as the transparent pigments in such a mulch-staining primer coat of the present invention.

The improved mulch modifying sprayable latex of this invention may also comprise high strength PU latex. Since one particular application of the improved mulch modifying sprayable latex of this invention is to bond mulch particles together into a mat strong enough to not be displaced by wind or rain, the high strength of PU latex after drying is highly desirable, because a thinner coating on the mulch particles is needed to achieve the desired bond strength between the mulch particles. Polyurethane dispersions are commercially available with strength >2000 psi and elongation to break after drying >400% (such as Witcobond W-281F or W-293, for example; also see U.S. Pat. No. 6,297,312 for an example of such a PU latex).

The improved mulch modifying sprayable latex of the present invention may also comprise micro-emulsion based primer coats to optimize the overall properties of the latex-modified mulch; said microemulsions are useful for sealing pores in mulch prior to application of a binding coat, to enhance bonding and to reduce usage of the binder latex, and such compositions are also useful for preserving mulch if the primer coats contain preservatives and UV blocking additives as described previously. Such microemulsion-based primer coats can be applied either before or after application of the mulch to the ground; the thus treated mulch particles may or may not be subsequently bonded together by a second adhesive latex. The primer coats can simultaneously serve several functions, for example pigmentation, resisting biodecay, protecting against UV bleaching, sealing the pores in the mulch so that subsequent treatment with a mulch-binding latex can be effective while using less total applied latex, and enhancing the bonding between the mulch-binding latex and the primer-treated mulch particles.

### EXAMPLES OF THE INVENTION

Table 1 shows a series of experiments in which a microemulsion latex (Joncryl 624) is added in varying proportions to a diluted PVAc latex (MB #1) at 11.5% polymer content, which is approximately the optimum concentration for spraying PVAc latex onto mulch for binding. The diluted PVAc latex was prepared by adding 175 ml of distilled water to 100 ml of PR-275M latex from Nanpao Chemicals in China (the as-received polymer content of the PR-275M latex was 32% by weight, with an additional 5.2% low molecular weight organics, including plasticizer, glycol, and coalescing solvent). Varying amounts of Joncryl 624 latex (48% solids, as supplied) were added to the diluted PVAc latex, then viscosity was measured, then the latex mixtures were subjected to freezing then thawing (one cycle), then the viscosity was re-measured.

TABLE 1
Brookfield Viscosity versus Blend Ratio of 12% solids PVAc latex + Microemulsion

| | | | | | | | | | | | | |
|---|---|---|---|---|---|---|---|---|---|---|---|---|
| diluted PVAc latex | 10 | 9.5 | 9 | 8 | 7 | 6 | 5 | 4 | 3 | 2 | 1 | 0 |
| Joncry1624 microemulsion | 0 | 0.5 | 1 | 2 | 3 | 4 | 5 | 6 | 7 | 8 | 9 | 10 |
| Weight % Joncryl 624 | 0% | 5% | 10% | 20% | 30% | 40% | 50% | 60% | 70% | 80% | 90% | 100% |
| initial viscosity (30 RPM) | 84 | 429.9 | 96 | 166 | 144 | 130 | 120 | 112 | 112 | 166 | 307.9 | 1096 |
| Viscosity after freeze-thaw | 1654 | 611.9 | 116 | 249.9 | 206 | 144 | 174 | 134 | 178 | 220 | 349.9 | 102 |
| Viscosity after F/T as % of initial | 1969% | 142% | 121% | 151% | 143% | 111% | 145% | 120% | 159% | 133% | 114% | 9% |

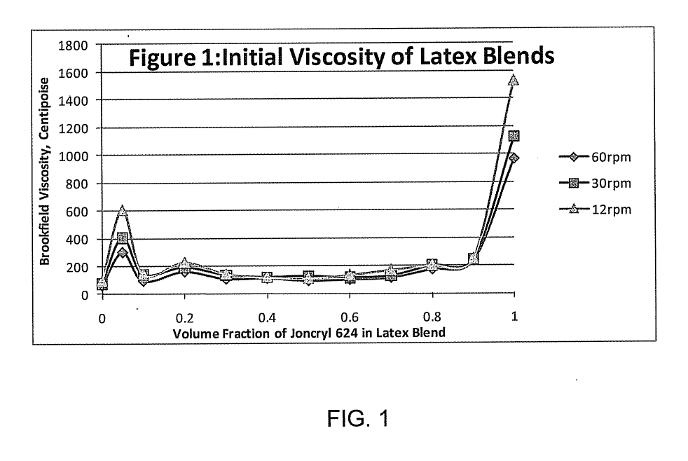

It is surprising that adding only 5% of microemulsion latex to the PVAc latex increased initial viscosity by a factor of five; see FIG. 1. (Note that since the polymer content of the Joncryl 624 latex was 48% by weight compared to 11.5% by weight for the diluted PR-275M latex, the peak viscosity at 5% Joncryl 624 corresponds to 18% Joncryl 624 based on the polymer content of the latex blend; subsequent experiments deal with this synergistic viscosity region in more detail.) At higher addition levels of Joncryl 624 (10% to 90%) the viscosity of the latex is reduced, and not changed much from the initial viscosity of the diluted PVAc latex. These surprising results were successfully replicated. It is apparent that there is an unexpected synergism at around 5% by volume Joncryl 624 in this experiment (about 18% Joncryl polymer by polymer mass ratio). The increase in viscosity is also expected to help suspend various other types of solid particles in the formulation, without requiring added hydrophilic or water soluble polymers to aid the suspension.

Table 1 shows that all the tested latex blend formulations (containing 5-90% of Joncryl 624) had better freeze-thaw resistance (in terms of viscosity stability during freeze-thaw) than either of the two latexes used to form the blend, as seen from the viscosity change due to freeze-thaw.

The next series of examples measures the compatibility of various desirable additives with the same diluted PVAc latex blend used in Table 1 and FIG. 1. Additives with insect repellant and/or cat repellant activity were tested; UV absorbing chemicals; pigments; biostatic chemicals; micronutrients; and latex viscosity modifiers and stabilizers were tested.

TABLE 2

| Stability Testing of Additivesinto 12% PVAc Diluted Latex | | | | | |
|---|---|---|---|---|---|
| (1 unstable, 5 stable) | Addition level, grams/100 ml | | | | |
| Ingredient | 0.25 | 0.50 | 0.75 | 1.00 | 1.25 |
| Joncryl 624 | 5 | 5 | 5 | 5 | 5 |
| Camphor oil | 5 | 4 | 4 | 3 | 3 |
| Eucalyptus oil | 5 | 5 | 5 | 4 | 3 |
| Citronella oil | 5 | 5 | 4 | 4 | 3 |
| Lemongrass oil | 4 | 4 | 4 | 3 | 2 |
| Red Iron Oxide | 4 | 4 | 3 | 2 | 2 |
| Zinvisable coated | 4 | 3 | 2 | 1 | 1 |
| Zinvisable | 5 | 5 | 4 | 4 | 4 |

In the tests of Table 2, the various ingredients were added either as powders or liquids directly to 8 ml of the same diluted PVAc latex blend which was also the PVAc latex portion of the binary blends of Table 1 and FIG. 1. Stability was evaluated after 2 days, then an additional 5 ml of distilled water was added. Stability was evaluated again after 9 additional days (11 days total, as a highly diluted, sprayable latex). A rating of 4 or 5 was judged to be readily sprayable. Any settling seen in rank 4 mixtures was readily re-dispersible with mild agitation. The liquid oily additives all separated over the eleven days with a visible oily layer floating on top; those with a compatibility rating of 3 did not cause any detectable coagulation of the remaining latex, whereas lemongrass oil caused some visible coagulation in the latex. The rankings of Table 2 reflect the stability after 11 days as described above.

Table 2 contains a few surprises. It is noteworthy that the coated grade of Zinvisible™ transparent zinc oxide caused the latex to coagulate, whereas the uncoated grade caused no problems within the 11 days observed, for levels up to 0.5 grams/100 ml of diluted PVAc latex, and only minor settling was observed for higher levels up to 1.25 parts per hindered parts of diluted latex. Also, the mixtures containing the microemulsion Joncryl 624 exhibited improved stability compared to the controls. Other nanosized UV absorbing pigments such as cerium oxide and various transparent iron oxides such as the Novant Chemicals Trionix transparent iron oxide pigments are also useful components of the mulch-holding latex of this invention.

To overcome the observed tendency of the odorant repellant oils to separate from the latex formulation we prepared an emulsion of the oils and water first using a simple oil soap (Delon Oil Soap) to form an emulsion. First, to test the suitability of Delon oil soap as an emulsifier for citronella and eucalyptus oil, we prepared a small quantity of emulsion by mixing 10 ml of water+0.5 grams citronella oil+0.5 grams of eucalyptus oil+0.15 grams of Delon Oil soap in a test tube, and shook this to obtain an emulsion. This emulsion was then placed in a lab centrifuge for ten minutes at high speed to look for evidence of separation; the emulsion was stable, with no evidence of separation.

Next, an actual formulation suitable for spraying and with improved stability was formed in this way:
1. An emulsion of 10% citronella oil and 10% eucalyptus oil was formed by mixing 5 grams of citronella oil+5 grams of eucalyptus oil+1 gram of Delon Oil soap and blending, then this was added to 39 grams of distilled water. Initially, this emulsion had two distinct layers, but then Cowles blade mixing was applied for one minute, which produced a stable Emulsion A. When this emulsion was centrifuged at 1000 RPM for 10 minutes, it separated into an oil layer and a water layer; however, when Emulsion A was later mixed into a variety of latex blends, all the blends were stable, and no oil was seen to separate as a film on top of the emulsions (as was seen in the oil/latex mixtures of Table 2), even after centrifuging.
2. A dilution of 10 ml of Joncryl 624+90 ml of distilled water was formed, this is Emulsion B.

3. 100 ml of PR-275M latex containing 32% polymer by weight was mixed with 100 ml of water; this is Emulsion C.

4. This blend was formed with mild agitation: 200 ml of Emulsion C was placed into a stirred beaker; then 55 ml of Emulsion B was added; viscosity was measured before and after this addition. This is Emulsion D. A sample was retained for stability testing.

5. Next, 127.5 ml of Emulsion D was mixed with 1.2 ml of Emulsion A+22 ml of distilled water to form Emulsion E. Viscosity was measured, and a sample of this was retained for stability testing.

6. Next, 50 ml of Emulsion E was placed into a stirring beaker, and 0.58 grams of Zinvisible was added with stirring. Viscosity was measured, and stability observations were made on this, which is called Emulsion F.

Emulsions B, C, D, and E were stable, after 10 minutes of centrifuging at 1000 RPM. Emulsion F had zinc oxide settling after centrifuging. Emulsions D, E, and F are examples of the present invention. Table 3 show viscosities versus shear rate for these three emulsions, prepared as above. Emulsion D shows clear and unexpected evidence of thixotropy, in that the viscosity increases at low speed. This is a desirable property for a sprayable emulsion, because the higher viscosity at low shear rate inhibits sedimentation and creaming. Note that no polymer viscosity modifiers have been added to Emulsion D.

Microemulsion latex particles are typically at least 10 times smaller than typical latex particles, which means there are at least 1000 times more latex particles per gram.

It is also noteworthy that Emulsion E, in which the oil concentrate Emulsion A is added to Emulsion D apparently disrupts the desirable thickening reaction between the microemulsion and PVOH. Since Emulsion A contains sodium laureth sulfate and other biodegradable organosulfate surfactants. Note that the data of Table 3 also show that the addition of zinc oxide caused a modest return of the thixotropic effect.

TABLE 3
Brookfield Viscosities of Emulsions C-F

| rpm | C | D | E | F |
|---|---|---|---|---|
| 1 | 360 | 1290 | 60 | 120 |
| 3 | 300 | 580 | 40 | 50 |
| 6 | 240 | 350 | 35 | 40 |
| 12 | 160 | 233 | 30 | 28 |
| 30 | 128 | 140 | 30 | 28 |
| 45 | 113 | 114 | 27 | 25 |
| 60 | 108 | 98 | 25 | 23 |
| 100 | 96 | 79 | 22 | 21 |

There is a fundamental difference between the emulsions of this invention and the prior art latex products in the market today for use in binding mulch particles together. Table 4 shows data from TGA (thermo-gravimetric analysis) applied to the basis formulation for the inventive sprayable mulch-holding formulations of the present invention (PR-275M latex diluted to 11.5% polymer content by weight), compared with un-diluted PR-275M, and a competitive latex product which is currently being sold by RS Industrial as “MulchHold.” The PR-275M latex contains glycols, coalescing solvents, and plasticizer to lower the minimum film forming temperature (MFFT) to approximately 7° Celsius, compared to about 15° Celsius for the competitive mulch-spraying latex Mulch-Hold from RS Industrial that was tested and is shown in Table 4 and FIG. 4 (a TGA curve). We also tested Rovace 117 from Rohm & Haas and found it to be quite similar to the Mulch-Hold product shown in Table 4.

There is no evidence that the RS industrial product contains desirable additives (plasticizers and glycols) to lower the MFFT and help coalescence. Lowering MFFT using specific examples of naturally occurring oils also works, and is novel.

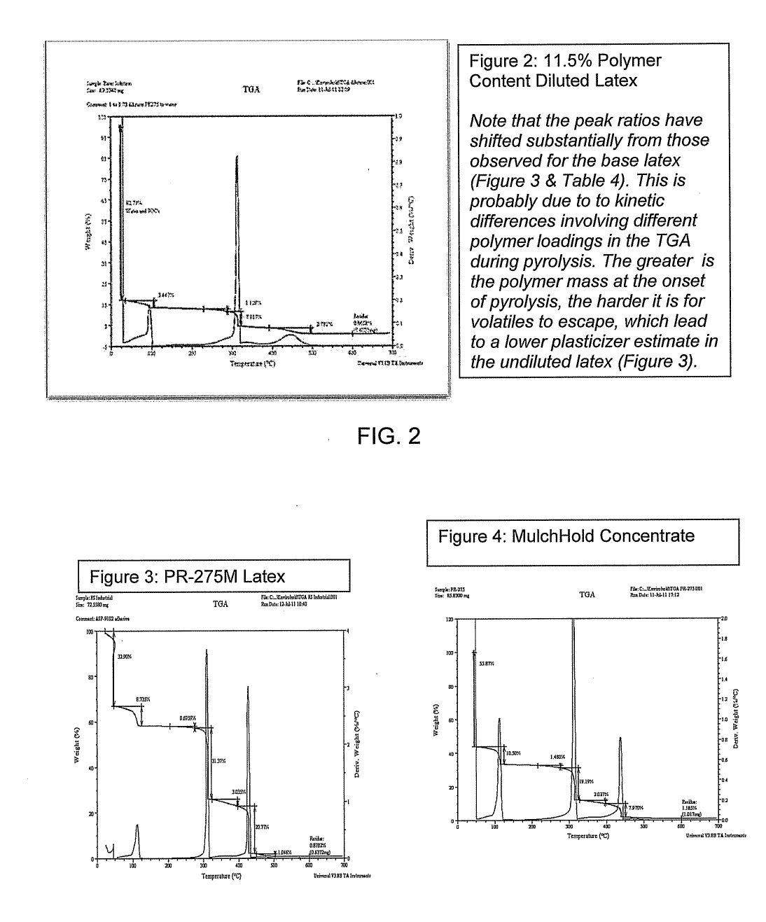

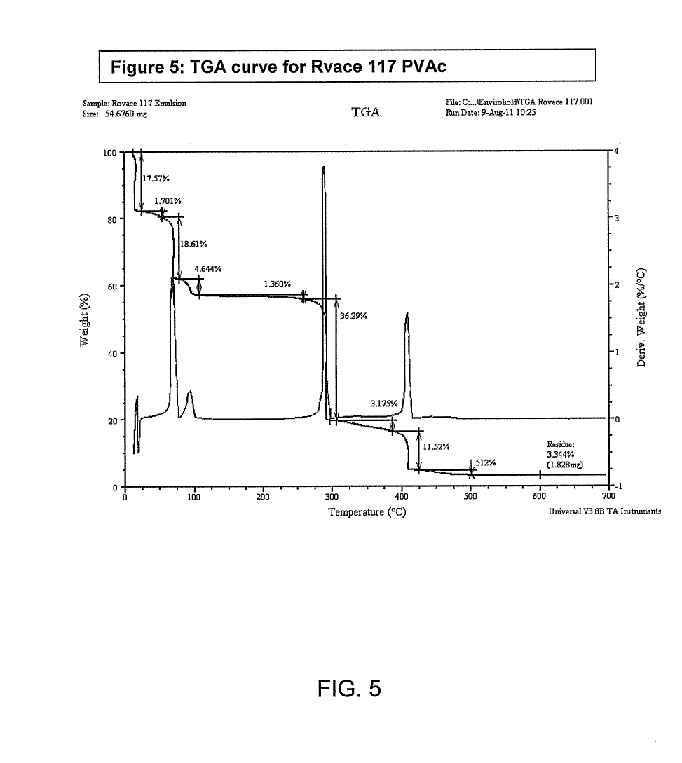

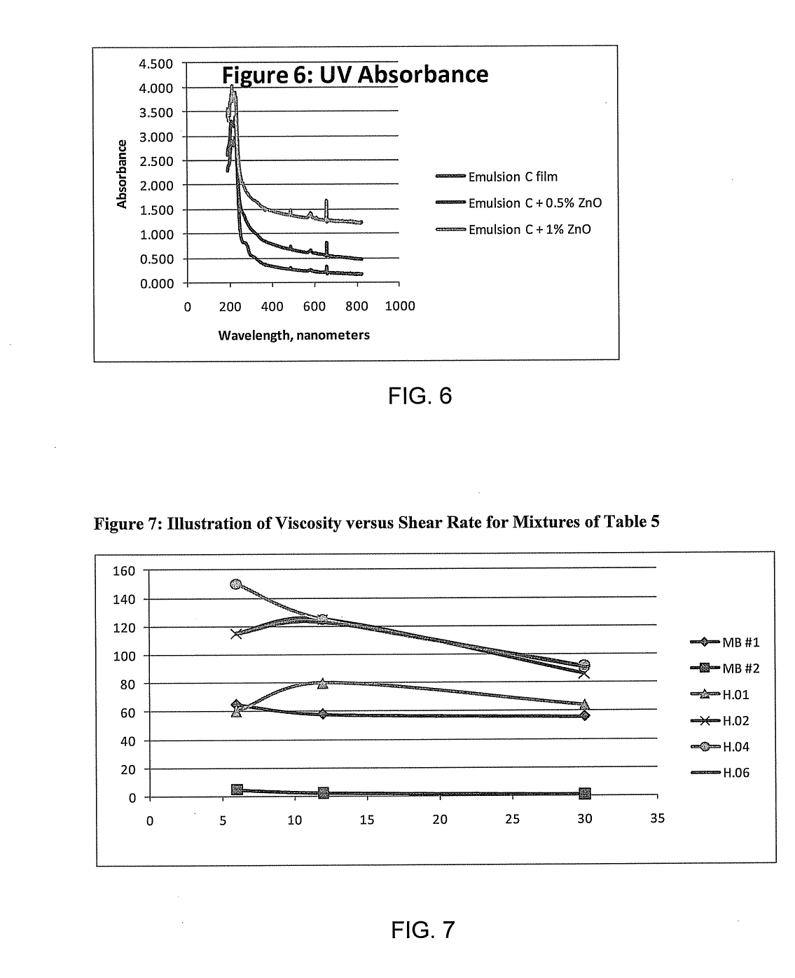

TABLE 4

| | | | TGA data on latexes | | | | | |
|---|---|---|---|---|---|---|---|---|
| | | | temp range, Celsius | | parts by weight out of 100 total | | | |
| | | Identification | | | PR-275 | PR-275 | Mulch-Hold | Rovace 117 |
| | | | low | high | diluted | as received | RS Industrial | Rohm & Haas |
| water + volatile solvent | | | 20 | 120 | 86.24 | 66.37 | 41.64 | 42.53 |
| coalescing solvent + glycols | | | 120 | 210 | 1.11 | 1.46 | 0.57 | 0.58 |
| | plasticizer | | 210 | 290 | 1.13 | 1.48 | 0.70 | 0.78 |
| loss of acetate from PVAc | | | 290 | 317 | 7.12 | 19.19 | 31.37 | 36.29 |
| | unknown | | 317 | 395 | 0.75 | 2.03 | 3.04 | 3.18 |
| Remaining PVOH? | | | 395 | 450 | 2.66 | 7.97 | 20.77 | 11.52 |
| | | | 450 | 500 | 0.13 | 0.40 | 1.05 | 0.51 |

| | | | | |
|---|---|---|---|---|
| 1500 + residue | 0.87 | 1.19 | 0.88 | 3.34 |

| FIG. # for TGA curve | | FIG. 2 | FIG. 3 | FIG. 4 | FIG. 5 |
|---|---|---|---|---|---|
| Non-volatile solids (to 317 C.): | | 11.52% | 30.69% | 57.10% | 56.12% |
| Total solids including plasticizer: | | 12.65% | 32.17% | 57.80% | 56.89% |
| Mass loaded into TGA (mg wet) | | 69.53 | 69.53 | 69.53 | 85.83 |
| Polymer mass in TGA sample: | | 8.01 | 21.34 | 39.70 | 48.16 |
| Weight % plasticizer in total solids: | | 8.92% | 4.60% | 1.20% | 1.37% |
| Weight % residue in non-volatile solids: | | 7.51% | 3.86% | 1.54% | 5.96% |
| Reported peaks total | | 98.8891 | 98.222 | 98.222 | 99.430 |
| | Unassigned total | 1.1109 | 1.778 | 1.778 | 0.5699 |

note 1:
Temperature increase pauses when greater than 1% loss of weight per minute
note 2:
Interpolated data are shown in italics

There are several observations that can be gleaned from Table 4. One thing to note is that the relative magnitude of the non-volatile residue (to 317° C.) scales as expected due to the original PR-275M sample being diluted in the ratio 100 latex:175 water, but that there are major shifts in the estimated levels of plasticizer and high temperature residues estimated in terms of weight % of total non-volatiles (to 317° C.) between measurements made on pure as-received PRM-275 latex and the diluted, ready-to-apply emulsion. These observed differences are believed to be due to limitations of the method that arise from having similar wet masses of the two latex samples, which implies significantly different non-volatile mass for the diluted versus non-diluted samples. Different masses in a TGA experiment are known to cause estimates of plasticizer content to shift due to more difficulty for the plasticizer to diffuse out of the polymer during the short time of the TGA test. The estimate of 8.92% plasticizer that was obtained on the diluted sample is believed to be more nearly correct, and this lines up well with the observed MFFT (7° C.) compared with information on the effect of plasticizer on MFFT of PVAc latex (Rohm & Haas datasheet on Rovace 117, 2006).

A mulch spraying test was done comparing the diluted PR-275M latex (11.5% non volatile solids) with equal solids content sprayable latex made by diluting the RS Industrial latex shown in Table 4 with water in the ratio of 100 parts latex to 395 parts water by volume (this also corresponds to 11.5% polymer solids in the sprayable latex). Superior mulch bonding was observed for a bed of cedar chips with the diluted PR-275M latex compared to the diluted RS industrial latex; this is believed to be due to lesser penetration into the much particles compared to the RS Industrial latex. We also expect that diluted PR-275 latex will be able to bind mulch together at lower temperatures than MulchHold, based on its lower MFFT.

The next series of experiments shows that it is possible to impart significant sunscreen activity to an improved, mulch modifying sprayable latex of this invention by incorporating nano-sized transparent metal oxide pigments. FIG. 2 shows UV absorbance of cast films derived from Emulsion C discussed above. All three films were transparent, even though the film cast from Emulsion C+1% by weight ZnO contains about 10% ZnO as a dry film. The particular grade of ZnO used in these experiments was Zinvisible™ from Horsehead Corporation of Monaca, Pa., USA; if the particles are well-dispersed the resultant films are transparent to visible light, but block UV light. Other known transparent metal oxides may also be used, including nanosized grades of cerium oxide, iron oxide, titanium dioxide, or any other metal which forms a nanosized, polymer-dispersible, non-water soluble metal oxide. These nanosized transparent oxides can be combined to enhance absorption of UVA and UVB radiation, as is known prior art in sunscreens that use synergistic combinations of ZnO and TiO₂. The same nanosized pigments that are helpful for absorbing UV light can also provide other useful functions, such as tinting the mulch (iron oxide, chromium oxide), or resisting biodecay (zinc oxide).

Next, experiments were done to better characterize the viscosity-building synergism between PR-275M latex and Joncryl 624 microemulsion (reported in Table 1 and FIG. 1). We created two diluted latex masterbatches, each containing 115 g/liter polymer content by weight:
MB #1 is 100 ml of PR-275M latex+175 ml of water
MB#2 is 20 ml Joncryl 624+67.6 ml of water

The polymer content of the latex used to make MB #1 was estimated from TGA (FIGS. 2 & 3) to be 11.52%, consistent with the theoretical polymer content based on the manufacturer's certification. The dilution of MB #2 to give 11.5% polymer content was based on the nominal solids of Joncryl 624 per the manufacturer. Table 5 gives the actual formulations mixed and the resultant viscosities at several shear rates, and the pH of each blend. Table 5 steps through concentrations of Joncryl 624 in latex blends with PR-275 PVOH-stabilized PVAc latex with Joncryl 624 concentrations ranging from 1% to 10% (based on polymer weight %). Table 5 clearly shows that there is an unexpected viscosity building synergism between these two latexes that can be seen at only 1% latex, and which is strong from 2-10% Joncryl in the blends.

FIG. 7 shows some of the data from Table 5 in a plot which makes it easier to recognize that there is an especially significant synergism between 4% Joncryl 624 and 96% PR-275M PVOH-stabilized PVAc latex (based on polymer content ratios). At a shear rate corresponding to 12 RPM, the three blends of Joncryl 624+PR-275M containing 2, 4, an 6% Joncryl 624 all had equivalent viscosity (˜125 cps, which is more than twice as high as the MB #1 control containing only PR-275M latex; however, at lower shear rate corresponding to 6 RPM in the Brookfield viscosity test, the 4% Joncryl 624-containing blend (H.04) had significantly higher viscosity than either the 2% Joncryl composition (H.02) or the 6% Joncryl composition (H.06).

TABLE 5
Viscosity Building Synergism

| 11.5% by Weigh Polymer Masterbatches | | | | | Measured using Kimax Mohr Pipets | | | |
|---|---|---|---|---|---|---|---|---|
| Ingredient | MB #1 | MB #2 | | | Temp 23.5 +/− 0.5 C. | | | |
| PR-275M | 100.00 | | | | Brookfield Viscosity | | | |
| Joncryl 624 | | 20.00 | | | Spindle #31, small s [symbol] | | LVDVII+ | |
| DI Water | 175.00 | 67.60 | | | spindle rpm (viscosity in centipoise (cp)) | | | |
| | | | | Compliance to ASTM E1293, Style I, Class B requirements | | | | |
| Sample | MB # 1 | MB #2 | total | Percentage | 6 | 12 | 30 | pH |
| MB #1 | | | | | 65 | 58 | 56 | 3.64 |
| MB #2 | | | | | 5 | 2.5 | 1 | 8.19 |
| H.01 | 19.8 | 0.2 | 20 | 1.00% | 60 | 80 | 64 | 3.51 |
| H.02 | 19.6 | 0.4 | 20 | 2.00% | 115 | 125 | 86 | 3.91 |
| H.03 | 19.4 | 0.6 | 20 | 3.00% | 135 | 118 | 85 | 4.09 |
| H.04 | 19.2 | 0.8 | 20 | 4.00% | 150 | 125 | 92 | 4.42 |
| H.05 | 19 | 1 | 20 | 5.00% | 125 | 118 | 90 | 4.59 |
| H.06 | 18.8 | 1.2 | 20 | 6.00% | 115 | 123 | 91 | 4.75 |
| H.07 | 18.6 | 1.4 | 20 | 7.00% | 115 | 123 | 89 | 4.91 |
| H.08 | 18.4 | 1.6 | 20 | 8.00% | 105 | 113 | 88 | 5.02 |
| H.09 | 18.2 | 1.8 | 20 | 9.00% | 115 | 115 | 87 | 5.15 |
| H.10 | 18 | 2 | 20 | 10.00% | 110 | 105 | 86 | 5.19 |

Mixed using a Thermolyne Maxi Mix II Type 37600 Mixer
Samples were contained in Blue Max 50 ml Polypropylene Conical Tubes [symbol]
indicates data missing or illegible when filed

The following experiments (Table 6) were aimed at a convenient method to obtain stable formulations of the present invention. The concept is to combine several novel features of this invention into a concentrated oil-based emulsion (Oil Mixture I or J) which can then be mixed with latex to simultaneously impart several desirable properties to an improved mulch modifying sprayable latex of this invention. First, a miscible mixture of essential oils and an emulsifier was made (Oil Mixture G); then this mixture was mixed with a transparent metal oxide (Oil Mixture H); then this mixture H was mixed with water in a high shear Cowles blade mixer to form Oil Mixture I, which was a stable emulsion with a soft settling property, which can be readily re-dispersed with mild stirring (no separation of an oily film on top was observed for either Oil Mixture I or Oil Mixture J). Finally, 10 ml of Joncryl 624 was added to Oil Mixture I to form Oil Mixture J. Mixtures of either Oil Mixture I or Oil Mixture J with PVAc latex results in examples of the novel improved mulch modifying sprayable latex of the present invention.

TABLE 6

| | Oil Mixtures | | | | | | | |
|---|---|---|---|---|---|---|---|---|
| | Oil Mixture G | | Oil Mixture H | | Oil Mixture I | | Oil Mixture J | |
| Ingedient | ml | grams | ml | grams | ml | grams | ml | grams |
| citronella oil | 50 | 43.25 | 50 | 43.25 | 50 | 43.25 | 50 | 43.25 |
| Eucalyptus oil | 50 | 46.125 | 50 | 46.125 | 50 | 46.125 | 50 | 46.125 |
| Biodegradable oil soap | 2 | 2 | 2 | 2 | 2 | 2 | 2 | 2 |
| Zinvisible ZnO | | | | 50 | 8.93 | 50 | 8.93 | 50 |
| water | | | | | 200 | 200 | 200 | 200 |
| Joncryl 624 | | | | | | | 59.22 | 62.18 |
| Specific Gravity (g/ml) | | 0.90 | | 1.27 | | 1.10 | | 1.09 |
| Weight % oils | | 97.8% | | 63.2% | | 26.2% | | 22.1% |
| Weight % non-volatile | | 100.0% | | 100.0% | | 41.4% | | 42.8% |

| | Sprayable latex mixtures | |
|---|---|---|
| | combine these ingredients: | |
| | Emulsion K | Emulsion L |
| Oil Mixture I: | 2 .10 | |
| Oil Mixture J: | | 2.48 |
| Rovace 117: | 10 | 10 |

| | | |
|---|---|---|
| water (so that final formulation contains 11.5% polymer): | 35.72 | 37.82 |

The oil-containing mixtures of Table 5 were prepared sequentially:
1. Oil Mixture G formed first: Citronella 50 ml+Eucalyptus oil 50 ml+biodegradable oil soap (Delon Oil Soap);
2. Zinc oxide added to Oil Mixture G using high shear Cowles blade mixer;
ground for 5 minutes at high speed to form oil mixture H.
3. Oil Mixture H (containing zinc oxide) was dispersed into 200 ml of water via Cowles blade mixing; grind for 5 minutes to form Oil Mixture I.
4. Mix 50 ml of emulsion I (54.9 grams) with 10 ml of Joncryl 624 to form Emulsion J. (although only part of I was used, the recipes of Table 5 were adjusted to show recipes on a consistent oil content basis).

Oil Mixtures G & H are illustrative; different essential oils may be substituted in place of citronella and eucalyptus, subject to the requirement that the selected oil mixture is compatible in the film forming polymer used in the final formulation at the final use level ratio of essential oils to polymers. Similarly different emulsifiers or mixtures of emulsifiers may be used to form the oil-in-water emulsions (of which Oil Mixture I and Oil Mixture J are merely illustrative examples); said emulsifiers can either be added to the oil mixture prior to emulsification (as shown in Table 6), or a water soluble emulsifier can be used, in which case it would be added to the water phase when creating the oil-in-water emulsions (similar to Oil Mixtures I and J)., Comparing the settling of I & J emulsions, it was obvious that Oil Mixture J experiences less settling of ZnO than Oil Mixture I; therefore the addition of Joncryl 624 has successfully reduced the settling of ZnO, and improved the stability of the ZnO/oil/water dispersion. Oil Mixtures I and J were subjected to 10 minutes treatment in a lab centrifuge operating at 1000 RPM; in both cases, the zinc oxide settled on the bottom of the centrifuge tubes; however, the sedimented ZnO found in the latex-containing Oil Mixture J after centrifuging was readily re-dispersed with mild agitation, whereas the zinc oxide formed a “hard cake” when Oil Mixture I was centrifuged.

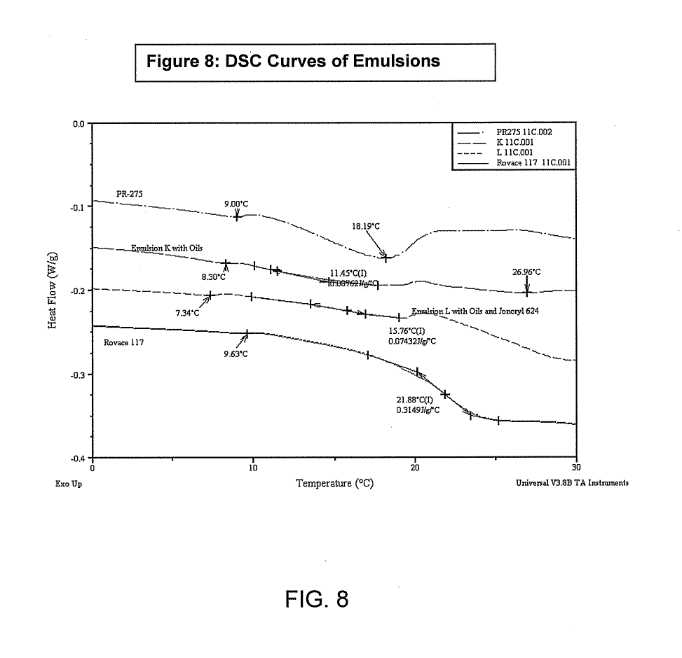

Emulsion K and Emulsion L (Table 6) are examples of the invention. Both of these latexes are based on Rovace 117 PVAc latex from Rohm and Haas (now a part of Dow Chemical) as the primary film forming polymer. The sample of Rovace we were working with had 56.12% polymer solids per a TGA experiment that was similar to FIG. 4 (not shown). Rovace 117 does not include any plasticizer, and as such it has an MFFT of 15° Celsius (similar to the MulchHold product from RS Industrial). Rovace 117 also contains PVOH as a stabilizer, but at a much lower (undisclosed) level than in PR-275 M latex. Emulsions K and L demonstrate that a mixture of plant-derived essential oils (in this case a mixture of citronella and eucalyptus oils, but other mixtures of naturally occurring oils or fragrances can also be used) can work to plasticize and lower the MFFT of PVAc latex (see FIG. 8, which is a differential scanning calorimetry curve of the resultant films from Emulsions K and L, the Rovace 117 control, and PR-275).

Emulsions K and L were placed in aluminum dishes, along with a Rovace 117 sample (each material in its own aluminum pan); these pans were placed into a refrigerator set at 11° Celsius overnight, with a small fan to facilitate drying. All three emulsions dried and formed compatible films overnight. This was somewhat surprising in the case of Rovace 117, for which the MFFT is 15° Celsius; theoretically, it should not have formed a film under these conditions, but it did. Note though that the conditions we employed were not identical to the MFFT test. FIG. 8 shows differential scanning calorimetry tests comparing dried films from Rovace 117, PR-275M (which is known to contain plasticizer), and Emulsions K and L. FIG. 8 shows that the citronella oil and eucalyptus oil that was incorporated into a Rovace 117-based formulation in Emulsion I at a 10 parts per hundred parts polymer basis have reduced the glass transition temperature from about 21.88° Celsius for Rovace 117 to about 11.45° Celsius. The formulation Emulsion J that also contains Joncryl 624 shows a Tg reduction to about 15.76° Celsius. The reduction of the first transition for the pre-plasticized latex PR-275M was 9° Celsius, 9.63° Celsius for the Rovace 117, 8.3° Celsius for Emulsion K, and 7.34° Celsius for Emulsion L.

It is noteworthy that there was no evidence of incompatibility of the essential oils with the PVAc latex after drying. The experiments of Table 6 also demonstrate that transparent metal oxide pigments can be incorporated into the emulsions of this invention. The particular method used to incorporate the metal oxide (transparent zinc oxide in this case) into Emulsions K and L via aqueous dispersions (Oil Mixture I and Oil Mixture J) is novel as well. Emulsion L also shows that addition of a microemulsion (Joncryl 624 in this case) improves settling resistance of a formulated sprayable latex for modifying mulch.

The viscosities of Emulsions K and L were lower than is desired for the sprayable emulsions of this invention. We believe this is because the Rovace 117 contains only a low level of PVOH soluble polymer for emulsion stabilization. Therefore, Emulsions K and L, while being examples of the invention, are less than optimal examples because of their low viscosity (<10 cps).

Insofar as it may be desirable to incorporate non-water soluble antioxidants, hindered amine light stabilizers, microbicides, and/or fungicides into the sprayable mulch-modifying latex formulations of this invention, the oil mixtures of Table 6 offer a methodology for incorporating these materials into the final formulation via a solution in the essential oils-containing phase. For example, antioxidant, organic UV absorbers, and oil-soluble HALS can be dissolved in the essential oils/plasticizer/surfactant mixture (Oil Mixture D) prior to emulsification in water. Then this oil-in-water emulsion can be mixed with polymer latex so that oil/plasticizer contents from 1-20% by weight of the polymer film can be attained after the polymer film is dried.

Next an experiment was performed to demonstrate that an emulsion of oil in water similar to Oil Mixtures I and J can be formed using only Joncryl 624 as an emulsifier. An aliquot of 30 ml of the MB #2 (20 ml Joncryl and 67.6 ml H2O) and mixed in 5 ml of citronella oil. This was mixed using high shear rate for 2 minutes using a Thermolyne Maxi-Mix with a Cowles-type mixing blade; a stable emulsion formed. The stability of this emulsion was tested by centrifuging at 1000 RPMs for 10 minutes; stability was good, only a slight color change was observed on the very top, but no distinct phase separation or line. When this emulsified oil/Joncryl emulsion was mixed at 4% of total polymer with PR-275 latex, the viscosity exceeded 100 cps.

1. An improved spray-on film-forming synthetic polymer latex formulation for protecting mulch from decay, solar bleaching, or displacement by wind or rain, in which the improvements include one or more of these features:
a. controlled wetting and penetration of the mulch,
b. increased life of the polymer film under direct sunlight exposure,
c. improved retention of color of the mulch due to a UV-blocking effect of the film-forming latex,
d. increased freeze-thaw stability of the latex,
e. improved shear stability of the latex formulation,
f. improved dilutability of the latex formulation,
g. ability of the latex formulation to act like a stain and dye the mulch,
h. controlled release of secondary and micronutrients, and/or
i. controlled release of pest control chemicals or odorants from the cured latex formulation.

2. An improved spray-on, film-forming synthetic polymer latex of claim 1 that includes wetting agents, dispersants, and/or microemulsions to control wetting and penetration of the mulch by the diluted latex.

3. An improved spray-on, film-forming, mulch-binding synthetic polymer latex of claim 1 that contains hindered amine light stabilizers to delay the breakdown of the film-forming synthetic polymer during exposure to sunlight.

4. An improved spray-on, film-forming, mulch-binding synthetic polymer latex of claim 1 that contains well dispersed, very small zinc oxide, iron oxide, and/or titanium dioxide particles, which are too small to scatter visible light to a significant extent so that the resultant film appears to be clear or translucent even though it contains enough zinc oxide, iron oxide and/or titanium dioxide to absorb at least a major portion of the ultraviolet light that is incident on the treated mulch particle.

5. An improved spray-on, film-forming synthetic polymer latex of claim 1 that contains well dispersed, very small UV-blocking nanoparticles derived from plant sap, which are too small to scatter visible light to a significant extent so that the resultant film appears to be clear even though it contains enough of the natural UV blockers to absorb at least a major portion of the ultraviolet light that is incident on the treated mulch particle.

6. An improved spray-on, mulch-penetrating, film-forming synthetic polymer latex composition of claim 1 that contains well dispersed, very small zinc oxide particles which slow biodecay of a sprayed mulch particle, and which are mixed with a microemulsion in which most of the polymer within the latex particles, by number ratio, is contained in latex particles smaller than 120 nanometers.

7. An improved spray-on, film-forming synthetic polymer latex stain of claim 1 that contains well dispersed, very small pigment particles which effectively dye a mulch particle when sprayed thereon, and which are mixed with of a microemulsion in which most of the polymer latex particles, by number ratio, are smaller than 120 nanometers.

8. An improved spray-on, film-forming synthetic polymer latex of claim 1 that has improved freeze-thaw resistance, due to formulation with a polymeric hydrogel and/or colloidal dispersion that inhibits agglomeration of latex particles during repeated freeze-thaw cycles.

9. An improved spray-on, film-forming, mulch-binding synthetic polymer latex of claim 8 in which the interaction of a solution of PVOH with a microemulsion leads to a thixotropic formulation with excellent antisettling behavior.

10. An improved spray-on, film-forming, mulch-binding synthetic polymer latex of claim 9 in which the particular microemulsion is Joncryl 624.

11. An improved spray-on, film-forming synthetic polymer latex of claim 1 that is more resistant to coagulation due to mechanical stirring than prior art PVAc emulsions used to stabilize mulch.

12. An improved spray-on, film-forming synthetic polymer latex of claim 1 that is more resistant to coagulation due to dilution than prior art PVAc emulsions used to stabilize mulch.

13. An improved spray-on, film-forming synthetic polymer latex of claim 1 that enables improved controlled release of secondary nutrients and micronutrients.

14. An improved spray-on, film-forming synthetic polymer latex of claim 1 that enables improved controlled release of cat-repelling chemicals.

15. An improved spray-on, film-forming synthetic polymer latex of claim 1 that enables improved controlled release of essential oils or other odorants.

16. An improved spray-on, film-forming synthetic polymer latex of claim 15 that enables improved controlled release of citronella, eucalyptus, tea tree, or neem oil.

17. An improved spray-on, film-forming synthetic polymer latex of claim 1 that enables improved resistance to creaming or separation in storage by also incorporating an anti-settling additive such as various polymers or colloids that give the water phase of the latex sufficient gel strength to stop Brownian motion and gravitational separation of the latex polymer particles.

18. An improved spray-on, film-forming synthetic polymer latex of claim 17 in which a yield stress is established through a synergistic interaction of a microemulsion with polyvinylalcohol dissolved in the water between the latex polymer droplets.

19. An improved spray-on, film-forming synthetic polymer latex of claim 2 that contains a microemulsion to enhance or control wetting and penetration of mulch.

20. An improved spray-on, film-forming synthetic polymer latex of claim 18 in which said microemulsion is a copolymer of styrene and butyl acrylate such as Joncryl™ 624.

21. An improved spray-on, film-forming synthetic polymer latex of claim 14 that contains renardine.

22. An improved spray-on, mulch-penetrating, film-forming synthetic polymer latex composition of claim 1 that contains well dispersed, very small zinc oxide, iron oxide, cerium oxide, and/or titanium dioxide nanoparticles which are small enough so as to not scatter visible light significantly, which block UV light and thus reduce UV decay of a sprayed mulch particle, and which are mixed with a microemulsion in which most of the polymer within the latex particles, by number ratio, is contained in latex particles smaller than 120 nanometers.

23. An improved spray-on, film-forming, mulch-binding synthetic polymer latex of claim 3 in which the hindered amine light stabilizers are not water soluble, but are soluble and dissolved in the oil mixture prior to the emulsification of the oil mixture, which then forms part of the total formulation.

24. An improved spray-on, film-forming, mulch-binding synthetic polymer latex of claim 20 in which the polymer weight percent of Joncryl 624 latex particles, as a fraction of total latex polymer content, is between 1-20%, more preferably 2-6%.
